(19) United States
(12) Patent Application Publication
(10) Pub. No.: US 2026/0158233 A1
(43) Pub. Date: Jun. 11, 2026

(54) Med Bed

(71) Applicant: Louis Hilliard, Powder Springs, GA (US)

(72) Inventor: Louis Hilliard, Powder Springs, GA (US)

(21) Appl. No.: 18/973,277

(22) Filed: Dec. 9, 2024

(51) Int. Cl. A61M 21/02; A61G 10/02; A61H 1/00; A61H 23/02; A61M 16/10; A61M 21/00; A61N 1/14

(52) U.S. Cl. CPC A61M 21/02; A61G 10/026; A61H 1/005; A61H 23/0236; A61M 16/1005; A61M 21/0094; A61N 1/14; A61G 2210/90; A61H 2201/0169; A61H 2201/0207; A61H 2201/10; A61H 2201/107; A61H 2201/5002; A61H 2201/5005; A61H 2201/5097; A61H 2230/065; A61M 2021/0055; A61M 2021/0066; A61M 2205/05; A61M 2205/42

(57) ABSTRACT

A Life Force MedBed Pod (herein referred to as a ‘pod’) is disclosed. The pod is comprised of several main parts: a chamber for patients; a computer with ‘life force’ software; a life force watch; a life force real time vitals mobile device software application; a Bluetooth surround-sound bar; a life force heated, massage and vibration mat; a set of grounding sheets; a frequency generator; an oxygen generator and body recharging rods. The pod combines five therapies that enhance sleep for the body, including but not limited to: frequency; electron donation; electro body balance; vibration and oxygenation. Other embodiments of the pod system can include a decompression option and a remote patient monitoring (RPM) system. An object of the invention is to enhance rest effectiveness and promote overall health.

### FIELD OF THE INVENTION

The present invention generally relates to rest. More specifically, it relates to an enclosed resting pod that enhances the resting experience.

### BACKGROUND

The first resting chambers open to the general public were introduced in Japan in the late 1970s and many were run as ‘capsule hotels.’ These chambers were personal pods, complete with TV and an alarm clock. Today, capsule hotels (also known as ‘pod hotels’) are enjoying a new wave of popularity around the world. Capsule hotels have also found homes at international airports. U.K. chain Hotel operates pods at London's Heathrow and Gatwick Airports as well as Amsterdam-Schiphol and Paris-Charles de Gaulle. Japan's First Cabin chain, with room sizes dubbed ‘First Class’ and ‘Business Class,’ opened in Osaka in 2008, expanded to Tokyo-Haneda Airport in 2012, and now boasts eight locations around the country. While such resting chambers have been growing in popularity, few are available in public places for hourly napping that focus on the use of visual and audio technologies for extreme relaxation. Some in the sleep industry have begun incorporating technology to improve rest in such pods. U.S. Pat. No. 8,337,385B1 disclosed a relaxation chamber with coordinating light and sound frequencies but does not incorporate internet connectivity. International Patent No. WO2018033837A1 teaches of a sleep-inducing chamber with controlled vibrations and sounds but does also not incorporate internet connectivity. United States Patent No. US20160287837A1 disclosed a resting pod for sleep and security that includes biofeedback mechanisms for aiding users in the inducement of sleep but does not include any forms of audio treatment. United States Patent No. US20190069685A1 disclosed a sleep chamber that includes a power-supply, vents, fans, speakers and lights but does not incorporate frequency treatments. What is needed is a holistic sleep pod that delivers multiple forms of sleep and health therapy in a single enclosure.

### SUMMARY OF THE INVENTION

The device herein disclosed and described provides a solution to the shortcomings in the prior art through the disclosure of an enclosed resting pod. An object of the invention is to promote safe rest, relaxation and naps. The resting pod accommodates a single person and has a bed, a locking enclosure with sound proofing, and a storage area for personal items securely. Such pods can be installed at home or even in crowded venues such as airports and train stations without requiring excess space. Each of these objects will be introduced below and will be discussed in further in the detailed description section of this document.

Another object of the invention is to allow a user to attain a deep level of rest within a limited time frame. The invention includes an internal, wraparound, digital ceiling screen and surround bar sound audio system allowing a user to immerse themselves in a virtual environment that includes a preset timer. Other embodiments of the pod system can include a MedBed Capsule comprised of a horizontal unit with room to lay down, a MedBed Chair that is compact with a sitting unit and a MedBed hyperbaric retrofit that allows the pod to selectively affix to existing hyperbaric chambers. Other embodiments of the pod system can include a decompression option that turns the pod into hyperbaric chamber.

Another object of the invention is to provide a body massage. The pod includes a life force heated, massage and vibration mat. The mat includes various intensity levels and tailored treatments for users. The vibrations form the mat excite all the cells in a user's body, bringing them back to life and breaks up old adhesions and muscle spasms along the neck, back and spine.

Another object of the invention is to provide a means to allow users understand the level of rest the invention provides. A biofeedback system is incorporated in the life force watch and allows users to quickly review their vital signs and sleep patterns at a glance. The watch can display a user's real-time vital statistics, including heart rate, blood pressure, oxygen levels, blood glucose, ECG records, and long-term sleep patterns, and communicate these statistics to the life force software;

Another object of the invention is to provide a means to allow users to control all aspects of the pod, including setting up therapy treatments, monitoring treatment activities and tracking medical histories on the pod computer. Anonymous forms of secured data can also be extracted and used for research purposes.

Another object of the invention is to provide users with frequency treatments. The pod includes a life force frequency generator with amplifier and software. The frequency amplifier quickly gets rid of health problems, parasites, pain, stress and the things in life that can drag a person down and them from feeling good and enjoying life. The pod is similar to a rife machine, chakra clearing and healing tone generator all in one.

Another object of this invention is to allow users to customize their resting experience by selecting rest scenarios on a proprietary cloud network service or via a participating third party. The resting pod visual and audio systems are connected to the internet and users can download preselected scenarios from outside vendors and store them on the cloud network. For example, a user preparing for a long trip at home prefers the sounds of waterfalls set to panoramic jungle scenery and imports a scenario from a third party onto the invention's proprietary website on the cloud server. When the user arrives at the airport and is waiting their flight, they can log into the resting pod system, select their customized scenario and begin their customized resting experience.

Another object of the aforementioned invention is to provide a means to allow users to adjust the climate within a resting pod. The pod includes a heater and an air conditioning unit that can be adjusted and even preprogrammed to sync with the aforementioned, customized video and audio scenarios. For example, those who prefer a tropical climate can raise the temperature to simulate a sunny warm environment.

Another object of the invention is to provide fresh oxygen to the user. The pod includes an oxygen concentrator that provides a constant flow of oxygen to users inside the pod. Supplemental oxygen can help relieve any problematic health symptoms. This therapy makes users feel more alert, helps them sleep better and places them in a better mood. Oxygen therapy can also allow users to do more activities such as traveling, including traveling to high altitudes.

Another object of the invention is to provide grounding for the user. The pod includes grounding sheets connected to grounding rods. Grounding is one of the best forms of electron donation. The life force grounding sheets add electrons to the body to help the user relax inside the pod.

It is briefly noted that upon a reading this disclosure, those skilled in the art will recognize various means for carrying out these intended features of the invention. As such it is to be understood that other methods, applications and systems adapted to the task may be configured to carry out these features and are therefore considered to be within the scope and intent of the present invention, and are anticipated. With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention. As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The objects features, and advantages of the present invention, as well as the advantages thereof over existing prior art, which will become apparent from the description to follow, are accomplished by the improvements described in this specification and hereinafter described in the following detailed description which fully discloses the invention, but should not be considered as placing limitations thereon.

### BRIEF DESCRIPTION OF THE FIGURES

The accompanying drawings, which are incorporated herein and form a part of the specification, illustrate some, but not the only or exclusive, examples of embodiments and/or features.

FIG. 1 shows the front view of the pod.

FIG. 2 shows a tope view of the pod.

FIG. 3 shows the life force watch.

FIG. 4 shows the computer tablet with life force software.

FIG. 5 shows the oxygen generator.

FIG. 5A shows the 4k Bluetooth surround-sound bar.

FIG. 6 shows the life force real time vitals software application.

FIG. 7 shows the life force heated, massage and vibration mat.

FIG. 8 shows the grounding sheets.

FIG. 9 shows a representative view of the inventive method.

FIG. 10 shows the RPM system.

Other aspects of the present invention shall be more readily understood when considered in conjunction with the accompanying drawings, and the following detailed description, neither of which should be considered limiting.

### DETAILED DESCRIPTION OF FIGURES

In this description, the directional prepositions of up, upwardly, down, downwardly, front, back, top, upper, bottom, lower, left, right and other such terms refer to the device as it is oriented and appears in the drawings and are used for convenience only; they are not intended to be limiting or to imply that the device has to be used or positioned in any particular orientation. Conventional components of the invention are elements that are well-known in the prior art and will not be discussed in detail for this disclosure.

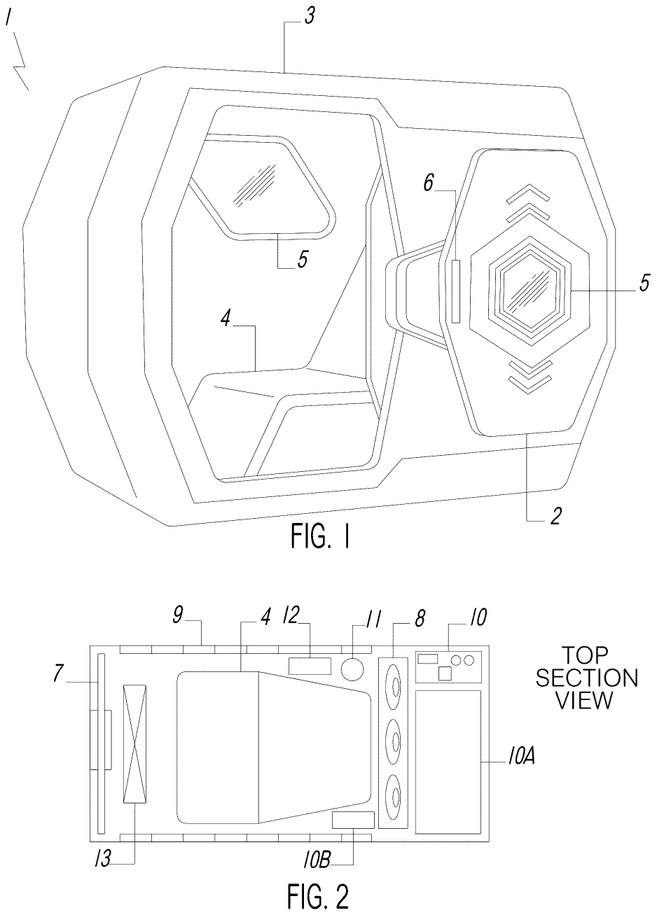

FIGS. 1 and 2 showing front perspective and top views of the preferred embodiment of the invention with slidable door 2 open and closed. The figures showing the invention being a rectangular-shaped pod 1 with said door 2 and enclosure 3 being made of a rigid material such as but not limited to metal, plastic and the like and containing a resting portion 4 (comprised of a chair, bed etc.) configured along its longitudinal axis. The invention also being configured to receive a resting user therein and may have a multitude of windows 5. Said door governed with but not limited to a pneumatic ram or cog assembly etc. and also being controlled manually by a user with handles 6 thereon. A secured storage area 10A is included on a distal portion to allow for personal belongings storage. Internal pod systems can include, but may not be limited to: an onboard widescreen video screen on a wall and the ceiling 7 (such as OLED, LCD etc.); sound bar with stereo, surround sound (Dolby etc.) 8; onboard specialty lighting and effects (LED etc.) 9; an onboard logic controller 10 having a central processing unit (CPU), micro controller with onboard memory, multimedia controllers, WiFi and onboard cellular transmitters and the like; onboard biofeedback sensors 11; ballistocardiograph and millimeter wave sensor 10B; onboard climate controls regulating hot and cold air (such as a rechargeable coolant system and an electric, radiant heating elements) and having a fan and air return plenum 13. All the aforementioned systems being connected to and controlled by said central processing unit as well as to an onboard A.C. power supply therein. Therapies performed inside the pod include but are not limited to: frequency therapy; grounding therapy; electro body balance therapy; massage/vibration therapy thermal therapy; oxygen therapy; and kinesiology testing; life force frequency healing; life force chakra balancing; oxygenated environment; sleep pattern analysis and grounding. Other embodiments of the pod can also include therapy addons such as: Ozone Therapy; Plasma Wand Therapy; Terahertz Therapy Cold Therapy (Cold Wraps); PEMF Blocker; Tesla Bio Healing; Aroma Therapy; Tesla Generation; and EMF Therapy (Beams).

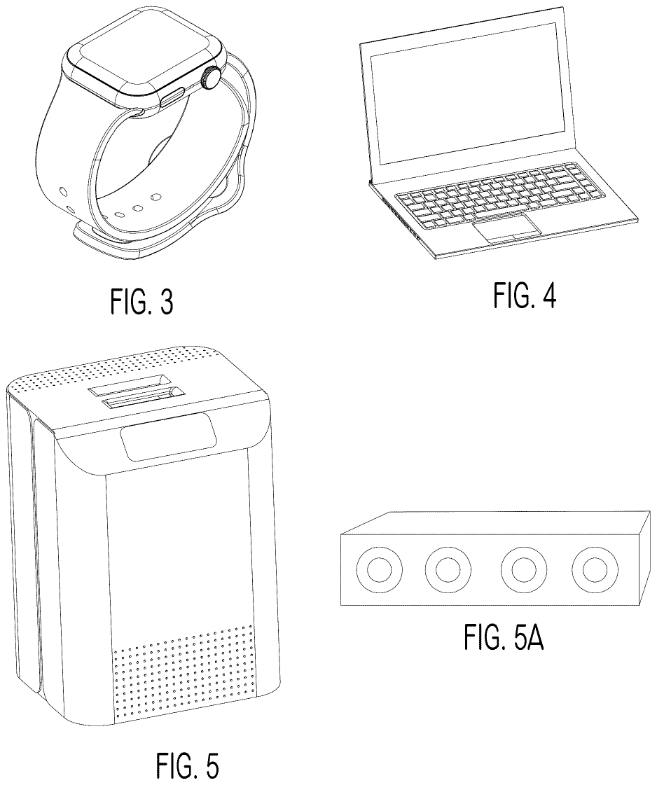

FIG. 4 illustrates the life force medbed computer. The computer may be comprised but is not limited to an laptop computer with Intel Core and can manage a user's entire health care plan, treatment, history, and communications using only one device and can also function as a design, edit, and work with a computer. The life force medbed computer controls all aspects of the pod including but not limited to the frequency generator, pod functions, monitoring health conditions and customizing all therapy programs. FIG. 3 showing the life force watch comprised of a digital wrist watch that communicates directly with the pod and the medbed computer. The watch allows users to obtain real time vital statistics including but not limited to: heart rate, blood pressure, blood oxygen levels, blood glucose, ECG records, and records long term sleep patterns. The watch also has a step counter. The watch system is able to scan users'vital signs in real time and record them on the life force software app and allows users to share vital signs and other health history with physicians through the app and software wirelessly.

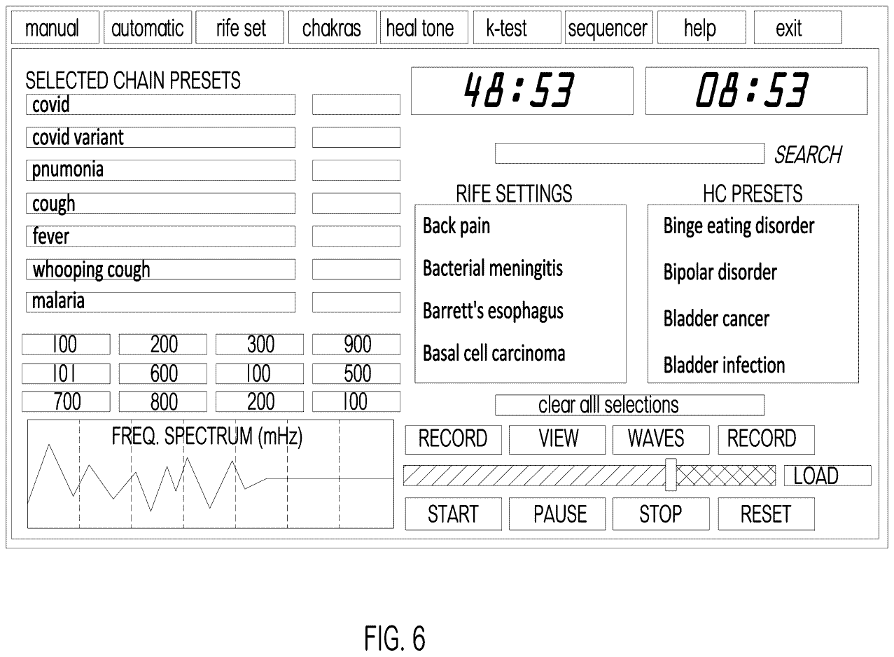

FIG. 5 illustrates the life force oxygen generator that pulls existing oxygen from the surrounding air and concentrates it. The unit achieves this task by removing nitrogen and other gases, as well as impurities, found in the air to purify it and compress it into medical-grade oxygen. Once the oxygen is sufficiently purifying and concentrated it is then delivered to the user inside the pod. The unit can provides from 1-7 Liters of adjustable pulse flow oxygen supply at a 93% concentration and can be adjusted with the life force computer or software application. FIG. 6 shows the life force frequency software and interacts with all pod therapies. The aforementioned software being written from code that may include, but not be limited to: Java, C++, Visual Basic, Linux, Basic and the like. The software also compatible with a plurality of operating systems such as, but not limited to: Windows, Apple, and Android, and compatible with a multitude of hardware platforms such as, but not limited to: personal desktops, laptops, tablets, smartphones and the like. Said software includes controlling configurations and settings for therapies that may include vibrations, patient vitals (pulse, pulse oximeter, blood pressure, temperature etc.) rife machines operations, frequencies and the like needed to treat various ailments as displayed on the screen's library listing. Other embodiments of the invention can include a system remote patient monitoring (RPM) system. The RPM is a podium that contains a remote monitoring system that can be transferred to several medbeds and can contain monitoring features and equipment such as but not limited to: blood sugar, pulse, body mass index, blood oxygen, spirometer, thermometer, and electrocardiogram. The RPM can schedule patient visits, perform prescriptions tracking and distribution, send medication reminders etc.

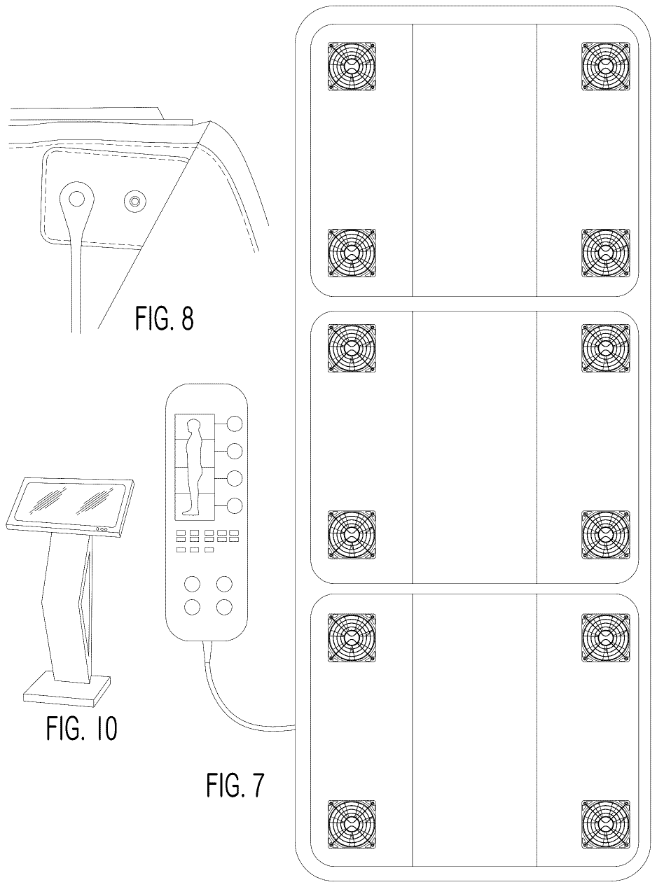

FIG. 5A illustrates the life force sound bar with built-in wireless Dolby Atmos^{TM} sound system and frequency amplifier that broadcasts specific healing frequencies. Embodiments of a speaker system include a linear array of speakers and a housing encasing a backside of the linear array of speakers. The speaker system further includes a pipe-mounting clamp coupled to the housing for mounting the speaker system to a surface. Embodiments of another speaker system include a linear array of speakers, a housing encasing a backside of the linear array of speakers, mounting brackets arranged along opposing lateral sides of the housing for mounting the speaker system to a surface, and a power cord extending from one of the opposing lateral sides of the housing. FIG. 7 showing the life force heated, massage/vibration mat that wakes up, and breaks up every cell in a user's body with the life force heated full body massage mat. The mat is engineered with three distinct intensity levels and five unique massage modes, providing a broad range of combinations. This customization allows for a fully personalized massage experience, tailored to meet the specific requirements and comfort levels of any individual user.

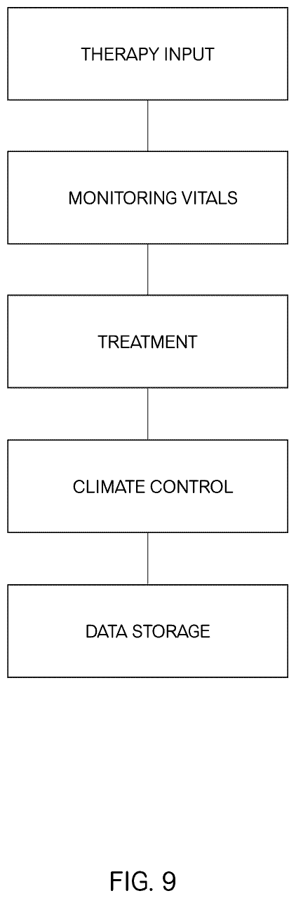

FIG. 8 shows the grounding sheet included with the pod with grounding cable affixed to a snap on said sheet that is then attached to a grounding. Said sheet comprises grounding devices and systems that conduct the earth's mobile free electrons from the earth to the body as if the user were standing barefoot on the earth. Further, the present invention relates to providing an abundant supply of free electrons to the body to reduce or prevent inflammation in the body. The invention generally comprises a body grounding device having a ground lead extending there from that is conductively coupled to a ground system which is connected to an earth ground anchor affixed to the pod. The grounding device comprises a grounding pad including a mesh layer substrate that contains between 10% and 20% carbon impregnated mono filament nylon fibers or between 1% and 10% silver coated mono filament nylon fibers. Preferably the mesh layer substrate comprises 95% nylon fibers and 5% silver coated nylon fibers. The fibers are knitted in a cross linking pattern throughout the fabric which allows a conductive connector to be easily placed in the device at any location to maintain conductivity with the fibers. A ground lead is conductively connected at one end to the conductive connector and at another end to an earth ground anchor. FIG. 9 showing a representative view of the inventive method that includes but is not limited to the following steps: receiving user input to configure a customized therapy program via a computer with life force software; monitoring real-time vital statistics using a life force watch and transmitting data to the pod system; providing an immersive audio and visual environment through a 4k Bluetooth surround-sound bar and internal digital screens; administering therapeutic treatments, including frequency therapy via a frequency generator and frequency amplifier, grounding therapy via grounding sheets and grounding rods, oxygenation therapy through an oxygen generator providing adjustable, purified oxygen flow, massage therapy through a heated, massage, and vibration mat with adjustable intensity, and electron donation therapy through body recharging rods; maintaining user comfort and environmental customization through integrated climate control systems; and collecting, analyzing, and storing user health and therapy data on a cloud network or onboard memory for feedback and research purposes.

It is additionally noted and anticipated that although the apparatus is shown in its most simple form, various components and aspects of the device may be differently shaped or slightly modified when forming the invention herein. As such those skilled in the art will appreciate the descriptions and depictions set forth in this disclosure or merely meant to portray examples of preferred modes within the overall scope and intent of the invention, and are not to be considered limiting in any manner. While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth.

What is claimed is:

1. A pod system for enhancing rest and promoting overall health, comprising:
a) a computer configured with life force software for controlling pod functions and therapy programs;
b) a life force watch configured to monitor and display a user's real-time vital statistics, including heart rate, blood pressure, oxygen levels, blood glucose, ECG records, and long-term sleep patterns, and communicate these statistics to the life force software;
c) a life force real-time vitals mobile device software application configured to store and share vital sign data;
d) a 4k Bluetooth surround-sound bar configured to provide immersive audio experiences and deliver frequency-based therapy through a frequency amplifier;
d) a life force heated, massage, and vibration mat with multiple intensity levels and massage modes tailored for user-specific requirements;
e) grounding sheets configured to connect to grounding rods and provide electron donation therapy to the user's body;
f) a frequency generator configured to emit therapeutic frequencies for health improvement and stress reduction;
g) an oxygen generator configured to purify and concentrate oxygen for user inhalation at adjustable flow rates;
h) a set of body recharging rods for delivering restorative therapy;
i) a central processing unit with onboard memory configured to control all pod functions, therapy configurations, and user settings; and
j) a secure enclosure with a resting portion, climate controls, a locking mechanism, and soundproofing, configured to provide an adjustable, immersive, and therapeutic environment for the user.

2. A method for enhancing rest and promoting overall health in a user utilizing a pod system, the method comprising the following steps:
a) receiving user input to configure a customized therapy program via a computer with life force software;
b) monitoring real-time vital statistics using a life force watch and transmitting data to the pod system;
c) providing an immersive audio and visual environment through a 4k Bluetooth surround-sound bar and internal digital screens;
d) administering therapeutic treatments, including:
a. frequency therapy via a frequency generator and frequency amplifier,
b. grounding therapy via grounding sheets and grounding rods,
c. oxygenation therapy through an oxygen generator providing adjustable, purified oxygen flow,
d. massage therapy through a heated, massage, and vibration mat with adjustable intensity, and
e. electron donation therapy through body recharging rods;
e) maintaining user comfort and environmental customization through integrated climate control systems; and
f) collecting, analyzing, and storing user health and therapy data on a cloud network or onboard memory for feedback and research purposes.

3. The pod system of claim 1, wherein the life force real-time vitals mobile device software application is a non-transitory computer-readable medium storing instructions executable by a processor to control a therapeutic pod system.

4. The pod system of claim 1, wherein the frequency generator emits a range of frequencies between 1 Hz and 10,000 Hz for therapeutic purposes.

5. The pod system of claim 1, further comprising a biofeedback system integrated into the life force watch for visualizing heart rate variability.

6. The method of claim 2, wherein the grounding therapy reduces inflammation by delivering a supply of free electrons to the user's body.

7. The medium of claim 3, wherein the instructions include integration with third-party cloud services to import and execute customized therapy scenarios.

8. The system of claim 1, wherein the secure enclosure includes a plurality of windows configured to allow natural light penetration while maintaining privacy.
